(19) United States
(12) Patent Application Publication
(10) Pub. No.: US 2026/0271936 A1
(43) Pub. Date: Sep. 17, 2026

(54) SURFACE SANITIZER WITH DUAL ACTION HYDROGEN PEROXIDE AND ALCOHOL

(71) Applicant: Medivators Inc., Minneapolis, MN (US)

(72) Inventors: Huyen Bui, Brooklyn Park, MN (US); Nam Bui, Brooklyn Park, MN (US); Kristopher Murphy, New Brighton, MN (US); Tuan Nguyen, Chaska, MN (US); Erin RODRIGUES, Maple Grove, MN (US)

(21) Appl. No.: 19/471,875

(22) PCT Filed: Apr. 16, 2024
(86) PCT No.: PCT/US2024/024713

§ 371 Date: Oct. 2, 2025

Related U.S. Application Data
(60) Provisional application No. 63/497,377, filed on Apr. 20, 2023.

(51) Int. Cl. A01N 59/00; A01N 25/02; A01N 31/02; A01P 1/00; A61L 2/22; A61L 101/02; A61L 101/34

(52) U.S. Cl. CPC A01N 59/00; A01N 25/02; A01N 31/02; A01P 1/00; A61L 2/22; A61L 2101/02; A61L 2101/34; A61L 2202/15

(57) ABSTRACT

Various examples disclosed relate to a sanitizing solution formulation. The present disclosure includes isopropyl alcohol in a range from about 70% to about 90% by weight of a first part, and hydrogen peroxide in a range of about 6% to about 8% by weight or less of a second part. The first part and the second part having a ratio of about 6:4 to a ratio of about 4:6 by volume. The first part and the second part are applied simultaneously to a surface to produce the sanitizing solution.

### CLAIM OF PRIORITY AND CROSS-REFERENCE TO RELATED APPLICATIONS

This application claims priority to U.S. Provisional Patent Application Ser. No. 63/497,377, filed on Apr. 20, 2023, entitled “SURFACE SANITIZER WITH DUAL ACTION HYDROGEN PEROXIDE AND ALCOHOL,” which is incorporated by reference herein in its entirety.

### BACKGROUND

Many scientific and medical environments require a certain amount of sanitizing. For example, clean rooms have stringent requirements for sanitized surfaces, so as to prevent contamination therein. A variety of sanitizing solutions are on the market and used to treat surfaces in such environments.

### SUMMARY OF THE DISCLOSURE

In an example, a sanitizing solution formulation can include isopropyl alcohol in a range from about 70% to about 90% by weight of a first part and hydrogen peroxide in a range of about 6% to about 8% by weight or less of a second part. The first part and the second part can have a ratio of about 6:4 to a ratio of about 4:6 by volume. The first part and the second part can be applied simultaneously to a surface to produce the sanitizing solution. The sanitizing solution can have a residual mass content of less than about 0.4 mg/g when assessed once applied to and dried on a surface.

In an example, a sanitizing solution formulation can include 40% isopropyl alcohol and 3% to 4% hydrogen peroxide. The formulation can have a volatile organic content (VOC) of less than about 50 wt. %, a mass residual content of less than about 0.3 mg/g, and a residual total organic carbon (TOC) content of less than about 0.1 mg/g.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various examples discussed in the present document.

FIG. 1 is a diagram depicting a spray bottle for application of the sanitizer in an example.

FIG. 2 is a bar chart depicting residual mass in an example.

FIG. 3 is a bar chart depicting residual TOC in an example.

FIG. 4 is a bar chart depicting stability of isopropyl alcohol (IPA) in an example.

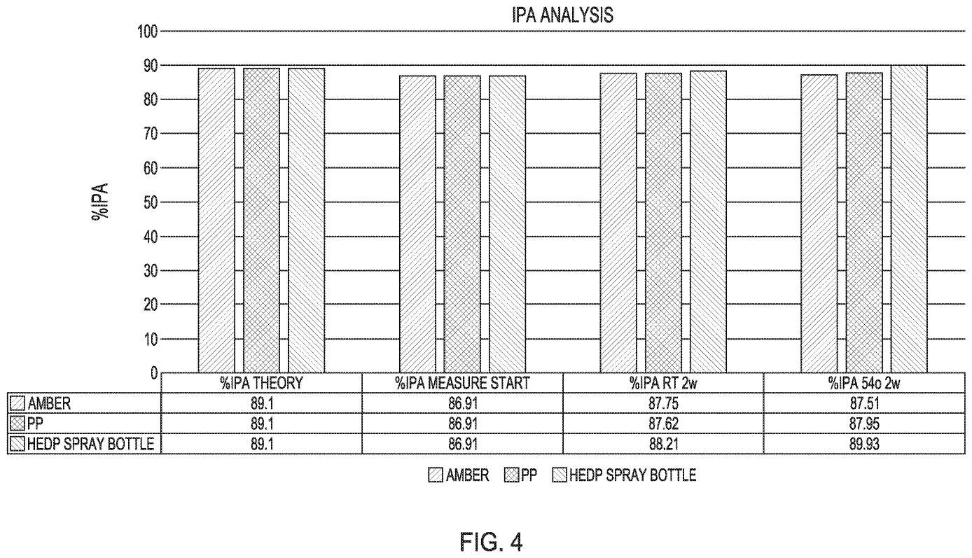

FIG. 5 is a bar chart depicting stability of hydrogen peroxide (H₂O₂) in an example.

FIG. 6 is a bar chart depicting stability of IPA under gamma radiation in an example.

FIG. 7 is a bar chart depicting stability of H₂O₂ under gamma radiation in an example.

### DETAILED DESCRIPTION

Reference will now be made in detail to certain examples of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Discussed herein is a formulation of a sanitizer solution including isopropyl alcohol (IPA) and hydrogen peroxide (H₂O₂). The solution is a spray solution of two parts: the first part is IPA 80-90 wt. %, and second part is H₂O₂ 6-7 wt. %. The spray is 50/50 by volume. The two parts are stored in separated halves of a container, and mixed when applied, such as by spraying. This allows for a longer shelf life of the sanitizing solution than pre-mixed formulations. The IPA and H₂O₂ formulation additional has low residuals and good efficacy.

Sanitizing solutions containing both hydrogen peroxide and alcohol can be volatile with a short shelf life. Moreover, alcohol such as IPA, while an effective sanitizer, is high in volatile organic carbon (VOC). For this reason, a sanitizer including a lesser amount of IPA, combined with H₂O₂, would be beneficial. The formulations discussed herein provide a sanitizer with 40% or less IPA, reducing the overall VOC of the formulation. The formulation described here also has a good shelf life and applicability, while maintaining efficacy.

### Definitions

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “solvent” as used herein refers to a liquid that can dissolve a solid, liquid, or gas. Non-limiting examples of solvents are silicones, organic compounds, water, alcohols, ionic liquids, and supercritical fluids.

The term “room temperature” as used herein refers to a temperature of about 15° C. to 28° C.

### Composition

Various sanitizing solution formulation herein include both isopropyl alcohol (IPA) and hydrogen peroxide (H₂O₂). The sanitizing solution, when used, can include about 40% isopropyl alcohol and 3% to 4% hydrogen peroxide.

The sanitizing solution formulation can include a first part containing the IPA, and a second part containing the H₂O₂. When applied to a surface, the first part and the second part can be mixed. The first part and the second part can have a ratio of about 6:4 by volume, a ratio of about 5:5 by volume, or a ratio of about 4:6 by volume.

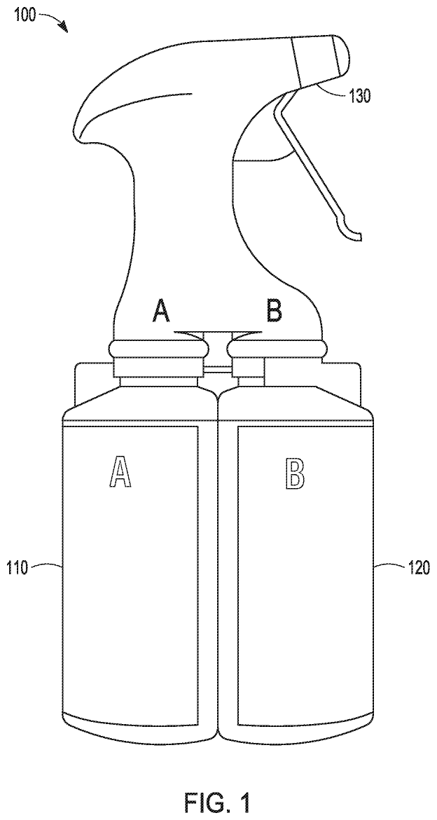

The first part and the second part can be applied simultaneously to a surface to produce the sanitizing solution. The first part and the second part can be stored in separated portions of a single container, such as discussed with reference to FIG. 1 below. The first part and the second part can be applied simultaneously by a sprayer to the surface.

The IPA can be in a range from about 70% to about 90% by weight of the first part, in a range of about 71% to about 89% by weight of the first part, in a range of about 72% to about 88% by weight of the first part, in a range of about 73% to about 87% by weight of the first part, in a range of about 74% to about 86% by weight of the first part, in a range of about 75% to about 85% by weight of the first part, in a range of about 76% to about 84% by weight of the first part, in a range of about 77% to about 83% by weight of the first part, in a range of about 78% to about 82% by weight of the first part, in a range of about 79% to about 81% by weight of the first part, or in a range of about 80% by weight of the first part.

The IPA can be about 30% to about 45% of the sanitizing solution when the first part and the second part are applied simultaneously, about 31% to about 44% of the sanitizing solution when the first part and the second part are applied simultaneously, about 32% to about 43% of the sanitizing solution when the first part and the second part are applied simultaneously, about 33% to about 42% of the sanitizing solution when the first part and the second part are applied simultaneously, about 34% to about 41% of the sanitizing solution when the first part and the second part are applied simultaneously, or about 35% to about 40% of the sanitizing solution when the first part and the second part are applied simultaneously.

The H₂O₂ can be in a range of about 6% to about 8% by weight or less of the second part, or in a range of about 6% to about 7% by weight or less of the second part.

The H₂O₂ can be about 3% to about 4% of the sanitizing solution when the first part and the second part are applied simultaneously, or about 3.2% to about 3.5% of the sanitizing solution when the first part and the second part are applied simultaneously.

The sanitizing solution can have a residual mass content of less than about 0.4 mg/g when assessed at least ten minutes following application to the surface. In an example, the sanitizing solution can have a residual mass content of less than about 0.4 mg/g, less than about 0.3 mg/g, less than about 0.2 mg/g, less than about 0.1 mg/g, or less than about 0.05 mg/g.

The sanitizing solution can have a residual total organic carbon (TOC) content of less than about 0.1 mg/g. In an example, the sanitizing solution can have a residual TOC content of less than about 01. mg·g, less than about 0.05 mg/g, or less than about 0.01 mg/g.

In an example, the sanitizing solution can have a volatile organic carbon (VOC) content of less than about 50 wt. %, or less than about 45 wt. %.

In an example, the sanitizing solution does not substantially degrade stainless steel, aluminum, polytetrafluoroelthylene, polyvinyl chloride, polypropylene, polycarbonate, polyethylene, polyvinylidene fluoride, or polyvinylidene difluoride when applied thereto.

In an example, the sanitizing solution, when used over a regular year period, does not substantially alter the mass, length, width, or thickness of stainless steel, aluminum, polytetrafluoroelthylene, polyvinyl chloride, polypropylene, polycarbonate, polyethylene, polyvinylidene fluoride, or polyvinylidene difluoride when applied thereto.

In an example, the sanitizing solution, when used over a regular year period, does not substantially visually alter surfaces of stainless steel, aluminum, polytetrafluoroelthylene, polyvinyl chloride, polypropylene, polycarbonate, polyethylene, polyvinylidene fluoride, or polyvinylidene difluoride when applied thereto.

In an example, the sanitizing solution, when used over a regular year period, does not substantially alter surfaces of stainless steel, aluminum, polytetrafluoroelthylene, polyvinyl chloride, polypropylene, polycarbonate, polyethylene, polyvinylidene fluoride, or polyvinylidene difluoride when applied thereto when subject to x-ray fluorescence (XRF) and Fourier transform infrared (FTIR) analysis.

In an example, the sanitizing solution can be effective at sanitizing Staphylococcus aureus, Tricophyton mentagrophytes, Trycophyton interdigitale, and Pseudomonas aeruginosa, in addition to herpes virus, adenovirus, and feline calicivirua, among other microorganisms. In an example, the santitizing solution passes when tested under AOAC Official Method 955.15: Testing Disinfectants Against Staphylococcus aureus, AOAC Official Method 955.17: Fungicidal Activity of Disinfectants using Tricophyton mentagrophytes (i.e. Trycophyton interdigitale), AOAC Official Method 964.02: Testing Disinfectants Against Pseudomonas aeruginosa, EN 13697, EN 14476, and ASTM E1053 protocols.

### Method of Making

The formulation discussed herein can be prepared in a variety of ways, such as by using any suitable preparation method. For example, the formulation can be made by combining the IPA and solvent in to a first part, and separately combining H₂O₂ and solvent into a section part. In each part, the components can be stirred or otherwise mixed.

In some cases, each part can be prepared as a pre-diluted formulation. In some cases, each part can be prepared undiluted. The first part and the second part can be stored separated from each other but combined when applied to a surface for sanitization.

### Method of Using

The sanitizing solution can be applied to a surface such that both the first part (including IPA) and the second part (including H₂O₂) are applied simultaneously to the surface.

FIG. 1 is a diagram depicting a spray bottle 100 for application of the sanitizer in an example. The spray bottle 100 has first half 110 and second half 120. Here, the first part of the formulation (e.g., including IPA) can be stored in the first half 110. The second part of the formulation (e.g., including H₂O₂), can be stored in the second half 120. The first part and the second part of the sanitizing solution can, in some cases, be pre-diluted.

When a user is ready to apply the sanitizing solution to a surface, they can apply the first part and the second part simultaneously. For example, in the spray bottle 100, this means the first part stored in the first half 110 and the second part stored in the second half 120 are simultaneously propelled to the sprayer 130 and sprayed on the surface together.

When sprayed together, the sanitizing formulation can include about 40% IPA and about 3-4% H₂O₂. Example 1 below depicts the application of such a combined spray.

### EXAMPLES

Various examples of the present disclosure can be better understood by reference to the following Examples which are offered by way of illustration. The present disclosure is not limited to the Examples given herein.

### Example 1. Formulation Spray Testing

Several Sample formulation sanitizers were produced and tested under ASTM e1560. The Samples included a Part A of isopropyl alcohol (IPA) and a Part B of hydrogen peroxide (H₂O₂). Part A was 80% IPA by weight, and Part B was 6% H₂O₂ by weight. The total spray formulation was produced at a 1:1 ratio of Part A to Part B. The Samples were produced with a goal amount of the two active components as follows:

$$\% {H}_{2} {O}_{2} = 1 * 6 / 1.8 = 3.3 3 \%$$

$$\% IPA = 1 * 0.8 * 80 / 1.8 = 35.5 \%$$

The actually produced Samples of Part A (IPA) and Part B (H₂O₂) are summarized in Table 1 and Table 2 below.

TABLE 1
Spray Sample IPA Content.

| | | Total | | | | | |
|---|---|---|---|---|---|---|---|
| | | Mass | | | | | |
| | Mass | with | | | Measured | | |
| Part A | sample | Butanol | Dilution | Peak Area | Concentration | % | % |
| (IPA) | (g) | (g) | Factor | IPA | (wt. %) | Target | error |
| Spray-1 | 0.4504 | 8.4885 | 18.84658 | 273240268 | 37.13 | 35.5 | 4.604898 |
| Spray-2 | 0.4549 | 8.513 | 18.714 | 264850790 | 35.74 | 35.5 | 0.679886 |
| Spray-3 | 0.4447 | 8.5239 | 19.16775 | 247435364 | 34.20 | 35.5 | −3.65976 |
| Spray-4 | 0.4485 | 8.4867 | 18.92241 | 268010386 | 36.57 | 35.5 | 3.015547 |

TABLE 2
Spray Sample H₂O₂ Content.

| Part B (H₂O₂) | weigh, g | Total, g | Dilute | Area peak | % H₂O₂ | % Target | % error |
|---|---|---|---|---|---|---|---|
| Spray-1 | 0.0893 | 0.9813 | 10.9888 | 744 | 3.30 | 3.33 | −1.01969 |
| Spray-2 | 0.091 | 0.9813 | 10.78352 | 762 | 3.31 | 3.33 | −0.48273 |
| Spray-3 | 0.0914 | 0.9819 | 10.74289 | 747.5 | 3.24 | 3.33 | −2.77252 |
| Spray-4 | 0.0906 | 0.9808 | 10.82561 | 736.4 | 3.21 | 3.33 | −3.50102 |

The produced Spray Samples were reproducible and had less than 5% error. To produce a final solution with IPA of about 40%, then Part A should be 90% of the formulation, and Part B should be about 5.4% of the formulation:

$$\% IPA = 0.8 * 90 / 1.8 = 40 \%$$

$$\% {H}_{2} {O}_{2} = 1 * 5.4 / 1.8 = 3 \%$$

This results in an effective disinfectant containing less than 8% hydrogen peroxide. Formulations with these respective amounts of Part A and Part B were further tested as discussed in the below Examples.

### Example 2. Formulation Residual Testing

The sample formulations were further tested for residuals compared to the Vesta-syde 1:128 SQ from Steris Life Sciences. The samples were tested under ASTME1560 protocols. Here, stainless steel beaker were used to test sample in a hood. A scale with five decimal points was used during testing (Mettler Toledo Model XPE205, SN B842601462). To analyze the total organic carbon (TOC) residuals, an M9 Total Organic Carbon (TOC) Analyzer (GE) was used. The samples tested are summarized below in Table 3:

TABLE 3
Samples tested for residuals.

| ID | H₂O₂ | IPA |
|---|---|---|
| Sample A | 3% | 40% |
| Sample B | 4% | 40% |
| Comparative Sample X | — | 70% |

Gravimetric Experiment. The samples were analyzed using gravimetric techniques. First, the stainless-steel beaker were weighed with the scale and recorded. The samples were then weighed and recorded. Approximately 50 g of each sample was used and added to a stainless-steel beaker. Each sample was tested three times (e.g., n=3). The test samples, in the stainless-steel beakers, were left in the hood until no more liquid was visually present. This was a time period of approximately two weeks. Then, after the evaporation, each of the stainless-steel beakers was weighed and recorded. The gravimetric residuals (WR) were calculated by the following method:

$$WR = WB 1 - WB$$

Where WB1 is the weight of the stainless-steel beaker after evaporation, and WB is the weight of the stainless-steel beaker before adding the Sample therein. The weight residual per gram sample (RM) was calculated as follows:

$$RM = WR / WS$$

Where WR is the gravimetric residuals and WS is the weight of the sample. The results of the gravimetric analysis are summarized below in Table 4:

TABLE 4
Gravimetric residual analysis.

| | | | | | | | Residual |
|---|---|---|---|---|---|---|---|
| | | | Tare | Sample | Sample | | mass |
| | | n | Mass (g) | Before (g) | After (g) | Residual (g) | (mg/g) |
| Sample 1 | 3% H2O2, | 1 | 72.85044 | 50.67232 | 72.85735 | 0.0069 | 0.052 |
| | 40% IPA | 2 | 73.90217 | 50.08047 | 73.90616 | 0.0040 | |
| | | 3 | 73.65112 | 50.03236 | 73.65597 | 0.0048 | |
| Sample 2 | 4% H2O2, | 1 | 72.82677 | 50.04630 | 72.83233 | 0.0056 | 0.051 |
| | 40% IPA | 2 | 73.77521 | 50.22226 | 73.78012 | 0.0049 | |
| | | 3 | 73.63054 | 50.19924 | 73.63541 | 0.0049 | |

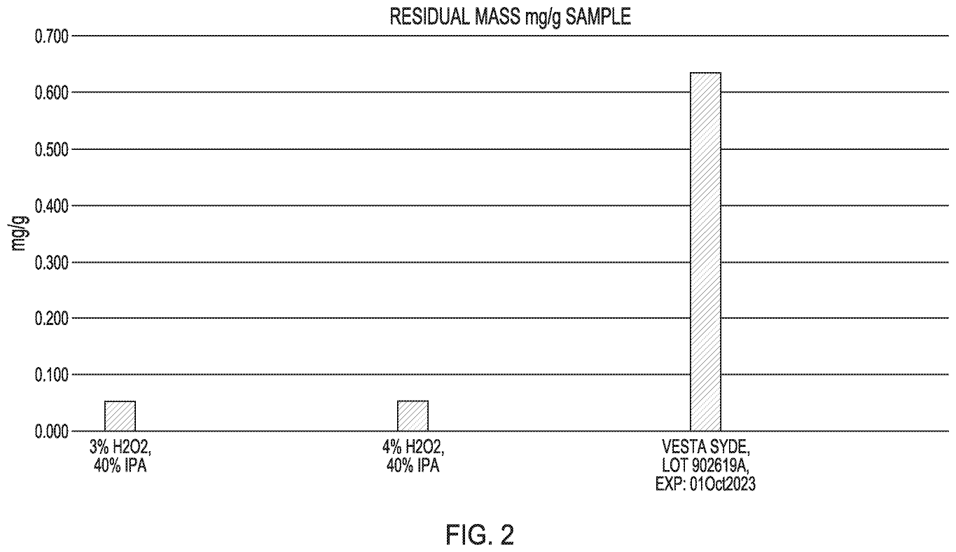

These results are also summarized in FIG. 2, a bar chart depicting residual mass in an example. Overall, the results showed a much lower residual mass, of about 0.051 to about 0.052 on average, for the inventive samples 1 and 2. This is compared to the Comparative Sample, with a residual mass of about 0.635 on average.

TO Measurements. The samples were also analyzed for TOC content. This total organic carbon method used the GE Sievers M9 Laboratory TOC Analyzer with accompanying GE Autosampler. The analyzer oxidized organic carbon in the samples with potassium persulfate and used an ultra-violet lamp to measure the CO₂ produced.

The method of the analyzer included: weighing and recording about 50 g of MilliQ water to samples from gravimetric experiment above, dissolving residuals by stirring with disposal pipet, and running with TOC with MilliQ water as a control. Next, the method included diluting samples to within appropriate concentration range (analyzer maximum was 50 ppm), and placing the diluted samples into 40 mL glass vials for TOC analysis (C&G Containers, P/N LFV040700005) with polypropylene seal caps. Each sample was run in triplicate.

Subsequently, the method “Testing Oxidizer at 3.4” stored on the Sievers M9 TOC Analyzer (S/N 15101095), was chosen. The method included the following parameters: oxidizer Rate or 3.4 μL/min and acid Rate of 1 μL/min. The residual TOC (mg/g of sample) was calculated as follows:

$$Residual TOC mg / g = ( Measure TOC mg / Kg \times Water dissolve ( g ) \text{:} 1000 g / Kg ) \text{:} WS$$

Where WS is the weight of the sample. The results of the TOC analysis are summarized in Table 5 below:

TABLE 5
TOC analysis.

| | | Weight | | | | | Total | Residual |
|---|---|---|---|---|---|---|---|---|
| | | Water to | Measured | Mass | Total | | TOC | TOC |
| | | Dissolve | TOC | Sample | Mass | Dilution | mg | mg/g |
| | | Residual (g) | ppm | (g) | (g) | Factor | residual | |
| Sample | 3% H2O2 | 50.1088 | 12.5 | 1 | 1 | 1 | 0.626 | 0.010 |
| A | 40% IPA | 50.0106 | 9.19 | 1 | 1 | 1 | 0.460 | |
| | | 50.0333 | 9.24 | 1 | 1 | 1 | 0.462 | |
| Sample | 4% H2O2 | 50.011 | 14.1 | 1 | 1 | 1 | 0.705 | 0.011 |
| B | 40% IPA | 50.8732 | 7.74 | 1 | 1 | 1 | 0.394 | |
| | | 50.2112 | 10.3 | 1 | 1 | 1 | 0.517 | |

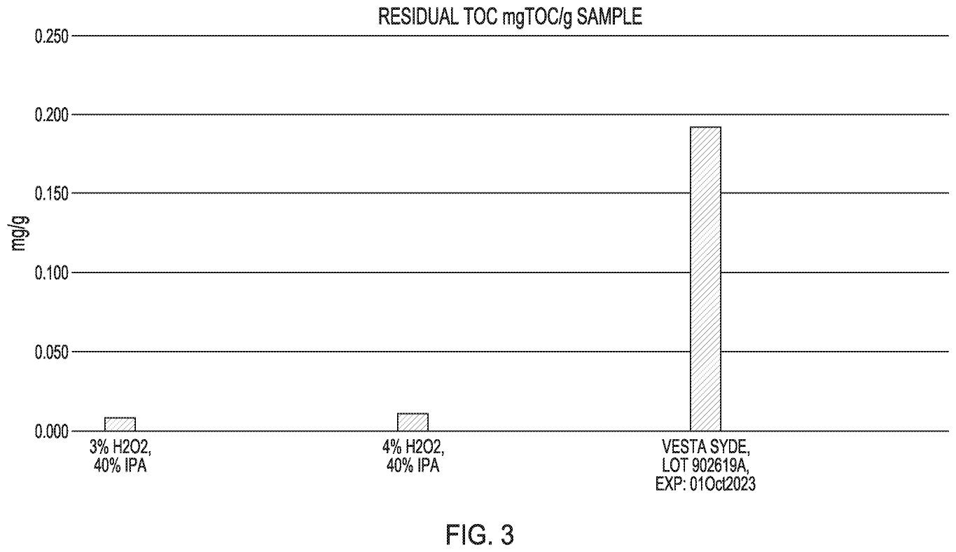

These results are also summarized in FIG. 3, a bar chart depicting residual TOC in an example. Overall, the inventive Samples A and B performed better than the Comparative Sample. The Samples A and B, on average, with 40% or less IPA and 3-4% H₂O₂, had a residual TOC content of about 0.010 mg/g to about 0.011 mg/g, compared to the comparative example with a residual TOC of about 0.192 mg/g. This is consistent with the gravimetric analysis. Overall, the inventive samples A and B performed well, with low residual content, under both testing procedures.

### Example 3. Formulation Efficacy Testing

The samples were additionally tested for efficacy. Here, several protocols were used: AOAC Official Method 955.15: Testing Disinfectants Against Staphylococcus aureus, AOAC Official Method 955.17: Fungicidal Activity of Disinfectants using Tricophyton mentagrophytes (i.e. Trycophyton interdigitale), AOAC Official Method 964.02: Testing Disinfectants Against Pseudomonas aeruginosa, EN 13697, EN 14476, and ASTM E1053. These various protocols were used to show the inventive sample sterilant formulation efficacy against a variety of microorganisms. The results of these tests are summarized below.

Table 6 depicts test results for the AOAC Official Method 964.02: Testing Disinfectants Against Pseudomonas aeruginosa. Here, the testing was carried out at 20° C. The samples used were 95% of normal concentration. The test time was 4 minutes and 45 seconds. The acceptance criteria under this protocol is ≤6/60 positive carriers.

TABLE 6
AOAC Official Method 964.02 Results.

| | | | P. aeruginosa | |
|---|---|---|---|---|
| Test No. | Sample ID | Chemistry | positive (4 m 45 s) | Pass/Fail |
| AOAC964.02-1 | Sample A | 3% H2O2, | 3/60 | Pass |
| | | 40% IPA | | |
| AOAC964.02-2 | Sample B | 4% H2O2, | 3/60 | Pass |
| | | 40% IPA | | |
| AOAC964.02-3 | Sample A | 3% H2O2, | 0/60 | Pass |
| | | 40% IPA | | |
| AOAC964.02-4 | Sample B | 4% H2O2, | 0/60 | Pass |
| | | 40% IPA | | |
| AOAC964.02-5 | Sample A | 3% H2O2, | 0/60 | Pass |
| | | 40% IPA | | |
| AOAC964.02-6 | Sample B | 4% H2O2, | 0/60 | Pass |
| | | 40% IPA | | |

Under the AOAC 964.02 protocol, the inventive Samples A and B passed all testing against P. aeruginosa.

Table 7 depicts test results for AOAC Official Method 955.15: Testing Disinfectants Against Staphylococcus aureus. Here, the test was carried out at 20° C. The samples were at 95% of normal concentration. The test time was 4 minutes and 45 seconds. The acceptance criteria under this protocol is ≤3/60 positive carriers.

TABLE 7
AOAC Official Method 955.15 Results.

| | | | S. aureus | |
|---|---|---|---|---|
| | | | positive (4 m | |
| Test No. | Sample ID | Chemistry | 45 s) | Pass/Fail |
| AOAC955.15-1 | Sample A | 3% H2O2, | 2/60 | Pass |
| | | 40% IPA | | |
| AOAC955.15-2 | Sample B | 4% H2O2, | 3/60 | Pass |
| | | 40% IPA | | |
| AOAC955.15-3 | Sample A | 3% H2O2, | 1/60 | Pass |
| | | 40% IPA | | |
| AOAC955.15-4 | Sample B | 4% H2O2, | 3/60 | Pass |
| | | 40% IPA | | |
| AOAC955.15-5 | Sample A | 3% H2O2, | 0/60 | Pass |
| | | 40% IPA | | |
| AOAC955.15-6 | Sample B | 4% H2O2, | 0/60 | Pass |
| | | 40% IPA | | |

Under the AOAC 955.15 protocol, the inventive Samples A and B passed all testing against S. aureus.

Table 8 depicts test results for EN 13697 test results for S. aureus. This protocol was conducted at 20° C. The test time was 4 minutes and 45 seconds.

TABLE 8
EN 13697 Results.

| | | | Contact | Log | |
|---|---|---|---|---|---|
| Test No. | Sample ID | Chemistry | time | reduction | Pass/fail |
| EN13697-1 | Sample A | 3% H2O2, 40% IPA | 4 min 45 sec | ≥6.48 | Pass |
| EN13697-2 | Sample B | 4% H2O2, 40% IPA | 4 min 45 sec | ≥6.49 | Pass |
| EN13697-3 | Comparative | 1% H2O2, 20% IPA | 4 min 45 sec | 2.14 | Fail |
| | Example Y | | | | |

Under the EN 13697 protocol, the inventive Samples A and B passed all testing against S. aureus. A Comparative Example Y, which had only 1% H₂O₂, did not pass.

Table 9 depicts test results for AOAC Official Method 955.17: Fungicidal Activity of Disinfectants using Tricophyton mentagrophytes (i.e. Trycophyton interdigitale). This protocol was conducted at 20° C. The test time was 4 minutes and 45 seconds.

TABLE 9
AOAC Official Method 955.17 Results.

| | | | T. interdigitale | |
|---|---|---|---|---|
| | | | positive | |
| Test No. | Sample ID | Chemistry | (4 m 45 s) | Pass/Fail |
| AOAC955.17-1 | Sample A | 3% H2O2, 40% IPA | 0 | Pass |
| AOAC955.17-2 | Sample B | 4% H2O2, 40% IPA | 0 | Pass |
| AOAC955.17-3 | Sample A | 3% H2O2, 40% IPA | 0/2 | Pass |
| AOAC955.17-4 | Sample B | 4% H2O2, 40% IPA | 0/2 | Pass |
| AOAC955.17-5 | Sample A | 3% H2O2, 40% IPA | 0/2 | Pass |
| AOAC955.17-6 | Sample B | 4% H2O2, 40% IPA | 0/2 | Pass |

Under the AOAC 955.17 protocol, the inventive Samples A and B passed all testing against T. interdigitale.

Table 10 depicts test results for virucidal testing. This protocol was conducted at 20° C. The test time was 5 minutes or 10 minutes, as shown in Table 10. ASTM E1053 protocols were used.

TABLE 10
ASTM E1053 virucidal testing results.

| | | | Contact | Log | |
|---|---|---|---|---|---|
| Sample ID | Chemistry | Virus | Time (min) | reduction | Pass/Fail |
| Sample A | 3% H2O2, | Herpes virus | 5, 10 | ≥4.75, ≥4.5 | Pass, Pass |
| | 40% IPA | | | | |
| Sample B | 4% H2O2, | Herpes virus | 5, 10 | ≥4.75, ≥4.5 | Pass, Pass |
| | 40% IPA | | | | |
| Sample A | 3% H2O2, | Adenovirus | 5 | 3.5 | Pass |
| | 40% IPA | | | | |
| Sample B | 4% H2O2, | Adenovirus | 10 | 5.25 | Pass |
| | 40% IPA | | | | |
| Sample A | 3% H2O2, | Feline | 5 | 3.5 | Pass |
| | 40% IPA | Calicivirus | | | |
| Sample B | 4% H2O2, | Feline | 10 | 3.75 | Pass |
| | 40% IPA | Calicivirus | | | |

Under the ASTM E1053 virucidal testing protocols, the inventive samples A and B passed when tested against the herpes virus, the adenovirus, and the feline calicivirus.

Table 11 depicts test results for EN 14476, which included cold temperature suspension testing results at 5° C. with S. aureus, at varying contact times.

TABLE 11
EN 14476 testing results.

| | | | Contact Time | Log | |
|---|---|---|---|---|---|
| Test No. | Sample ID | Chemistry | (min) | reduction | Pass/Fail |
| EN14476-1 | Sample A | 3% H2O2, | 30, 60, 120, | ≥6.36 for | All Pass |
| | | 40% IPA | 180 | all | |
| EN14476-2 | Sample B | 4% H2O2, | 30, 60, 120, | ≥6.36 for | All Pass |
| | | 40% IPA | 180 | all | |
| EN14476-3 | Comparative | 70% IPA | 30, 60, 120, | ≥6.36 for | All Pass |
| | Example | | 180 | all | |

Under the EE 14476 protocol, the inventive Samples A and B passed under all contact times.

Overall, the inventive samples A and B, containing 3-4% H₂O₂ and 40% IPA, performed well against a broad array of microorganisms, and performed the same or better than comparative examples containing less H₂O₂ or more IPA.

### Example 4. Formulation Material Compatibility

The samples were additionally tested for material compatibility. Under these tests, various materials were soaked in the inventive sanitizer for a period of time and analyzed for a change in weight, dimension, appearance, and chemical structure. For context, in a clean room setting, surfaces are sanitized sixteen times a day (e.g., every thirty minutes over an 8 hour workday). Over a year, this results in the equivalent of 14.4 days of the surface being soaked in sanitizer. Common clean room materials that were tested for compatibility with the inventive sanitizer formulations include stainless steel, aluminum, Teflon®, acrylic, polyvinyl chloride, polypropylene, polycarbonate, polyethylene, PVDF, and Tygon tubing.

The samples tested on these various surfaces are summarized in Table 12 below:

TABLE 12
Samples tested for material compatibility.

| Sample ID | H₂O₂ | IPA |
|---|---|---|
| Sample A | 3% | 40% |
| Sample B | 4% | 40% |
| Comparative Sample X | — | 70% |

The samples were tested for 12-month equivalent materials compatibility by a 14-day soaking. The results are summarized below in Table 13. All values in Table 13 are shown as percentage differences from original measurements compared to measurements done after the 14-day soak. The measurements included mass, length (or outer diameter, OD), width (or inner diameter, ID), and thickness.

TABLE 13
Materials compatibility data.

| | | | | | Sample A and Sample B | | | |
|---|---|---|---|---|---|---|---|---|
| | Comparative Sample Z (70% IPA) | | | | (3-4% H₂O₂, 40% IPA) | | | |
| | | OD or | ID or | Thick- | | OD or | ID or | Thick- |
| | Mass | Length | Width | ness | Mass | length | width | ness |
| | % | % | % | % | % | % | % | % |
| Material | Change | Change | Change | Change | Change | Change | Change | Change |
| Stainless | 0.00 | 0.00 | 0.13 | −1.24 | −0.01 | −0.04 | 0.08 | −0.97 |
| Steel 304 | | | | | | | | |
| Aluminum | 0.00 | −0.02 | 0.00 | 0.00 | −0.01 | 0.03 | 0.08 | 0.21 |
| 2024 Bare | | | | | | | | |
| Teflon | −0.02 | 0.00 | 0.07 | NA | −0.03 | 0.00 | 0.16 | NA |
| Acrylic | 0.80 | −0.55 | 3.99 | NA | 1.01 | 1.42 | −0.12 | NA |
| Polyvinyl | 0.31 | 0.04 | −0.16 | 0.22 | 0.33 | −0.03 | 0.80 | 0.20 |
| Chloride | | | | | | | | |
| Polypropylene | −0.04 | 0.00 | 0.51 | NA | −0.11 | −0.14 | −0.02 | NA |
| Polycarbonate | 0.26 | 0.04 | −0.05 | 0.01 | 0.71 | 0.04 | 0.16 | −0.44 |
| Polyethylene | 0.50 | 0.00 | 0.10 | 0.22 | −1.45 | 0.08 | 0.82 | 0.22 |
| PVDF | NA | NA | NA | NA | 0.00 | 0.02 | 0.08 | −0.21 |
| Tygon | −3.76 | −2.14 | −0.87 | −0.34 | −2.99 | 0.36 | −1.50 | −0.33 |

As shown in Tables 13, quantitative analysis showed good material compatibility across all types with the inventive Samples A and B. The Samples A and B performed as well if not better than the Comparative Example Z.

Additionally, the materials were analyzed under x-ray fluorescence (XRF) and Fourier transform infrared (FTIR) after soaking. These results are shown below in Table 14:

TABLE 14
Materials compatibility XRF/FTIR data.

| | Comparative Sample Z | Sample A and Sample B |
|---|---|---|
| | (70% IPA) | (3-4% H₂O₂, 40% IPA) |
| Materials | XRF/FTIR Inspection | XRF/FTIR Inspection |
| Stainless Steel | No change | No change |
| Al | No change | No change |
| Teflon | No change | No change |
| Acrylic | No change | No change |
| Polyvinyl Chloride | No change | No change |
| Polypropylene | No change | No change |
| Polycarbonate | No change | No change |
| Polyethylene | No change | No change |
| PDVF | No change | No change |
| Tygon | No change | No change |

Additionally, after soaking, the materials were analyzed visually, summarized in Table 15:

TABLE 15
Materials compatibility visual inspection.

| | Comparative Sample Z | Sample A and Sample B |
|---|---|---|
| | (70% IPA) | (3-4% H₂O₂, 40% IPA) |
| Materials | Visual Inspection | Visual Inspection |
| Stainless Steel | No change | No change |
| Al | No change | No change |
| Teflon | No change | No change |
| Acrylic | Sticky, Cloudy | Sticky, Cloudy |
| Polyvinyl Chloride | No change | No change |
| Polypropylene | No change | No change |
| Polycarbonate | No change | No change |
| Polyethylene | No change | No change |
| PDVF | No change | No change |
| Tygon | No change | No change |

As shown in Tables 14 and 15, both XRF/FTIR data and visual inspection showed good material compatibility across all types with the inventive Samples A and B. The Samples A and B performed as well if not better than the Comparative Example Z.

### Example 5. Formulation Stability Testing

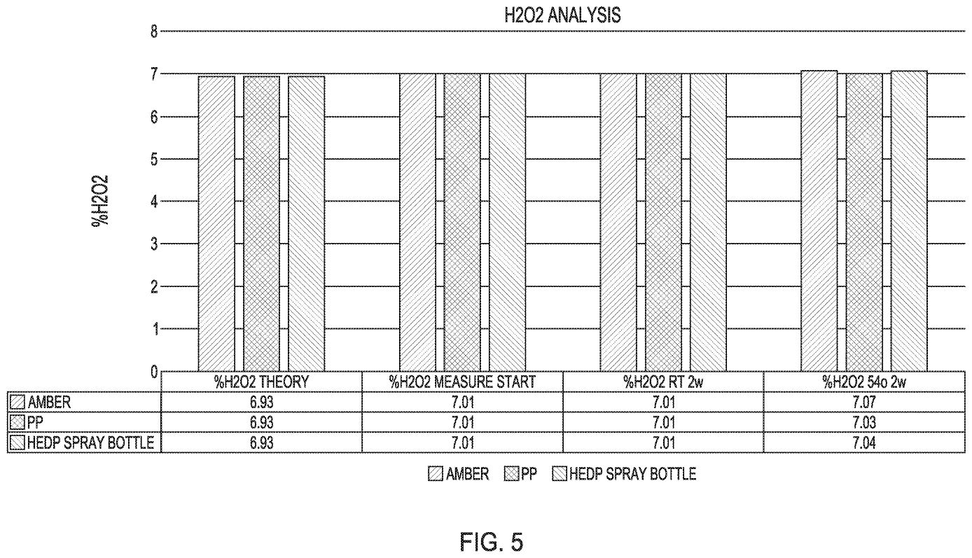

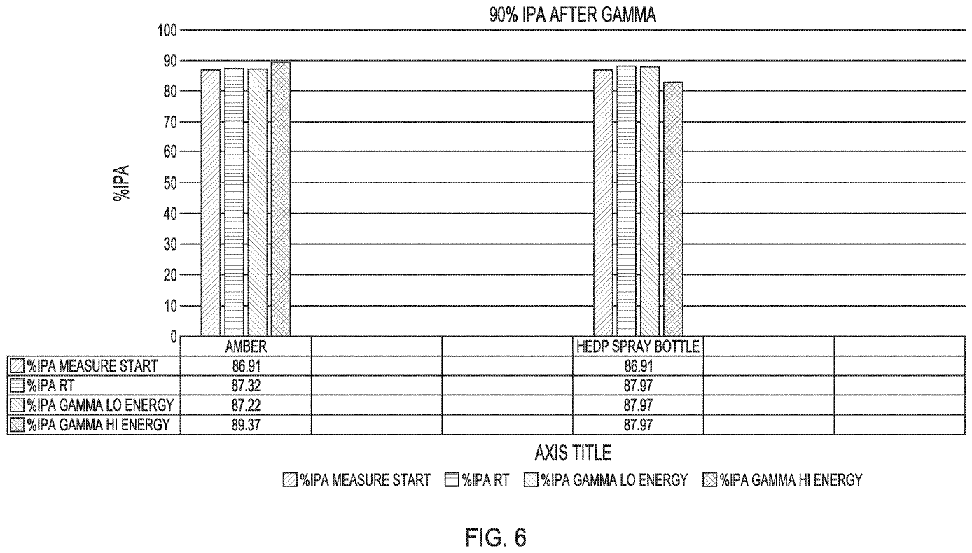

The samples were additionally tested for stability under gamma sterilization and radiation. Here, a sample with 90% IPA and 7% H₂O₂ was tested for solution stability with a gamma sterilization test and at 54° C. for two weeks. The stability of IPA is depicted in FIG. 5. The stability of H₂O₂ is depicts in FIG. 6. Overall, the formulation with 90% IPA and 7% H₂O₂ had good stability.

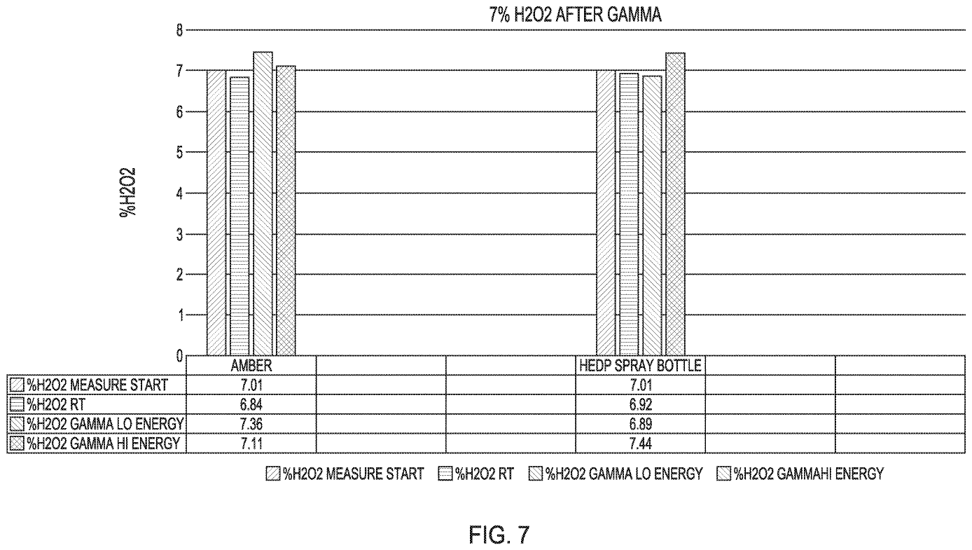

The sample with 90% IPA and 7% H₂O₂ was tested for gamma radiation at a high dose and a normal dose. FIG. 7 is a bar chart depicting stability of IPA under gamma radiation doses. FIG. 8 is a bar chart depicting stability of H₂O₂ under gamma radiation doses. Overall, the formulation with 90% IPA and 7% H₂O₂ had good stability.

### Example 5. Formulation Volatile Organic Carbon (VOC) Testing

The samples were additionally tested for volatile organic carbon (VOC) content. IPA is the only component of the formulation that contributes to the level of VOC. Thus, reduced IPA would lower VOC. Hydrogen Peroxide has no carbon, and does not produce VOC. For this reason, a lower IPA formulation produces less VOC.

The inventive samples A and B with 3-4% H₂O₂ and 40% IPA were tested compared to the comparative sample Z containing 70% IPA. First, an aluminum foil weighing dish (Aluminum dish cat. No 08-832-110), was weighed (W1) and recorded. Then, about 3 g of the sample was placed into the weighing dish. The weight, Ws, was recorded. The sample stood for 1 hour at ambient.

Subsequently, the aluminum foil dishes containing samples were heated in the force draft oven (Model 48800 Barnstead/Thermodyde) for 60 min at 110+/−5° C. Adequate ventilation was provided. The dish was removed from the oven and placed immediately in a desiccator. The dish was cooled to ambient temperature and weighed to within 0.00001 g (Mettler Toledo Model XPE205 SN B842601462), W2. This analysis was done in duplicate sets.

The % of all volatile components, Wv, was calculated as follows:

$$Wv = ( W 2 - W 1 ) * 100 / Ws$$

The amount of water (Ww) in formula was calculated with the following:

$$70 \% IPA have Ww = 30 \times 100 / ( 0.786 * 70 + 30 ) = 35.28 \%$$

$$3 \% H2O2 , 40 \% IPA have Ww = ( 100 - 3 - 40 ) = 57 \%$$

$$4 \% H2O2 , 40 \% IPA have Ww = ( 100 - 4 - 40 ) = 56 \%$$

Using this information, the percent of VOC was calculated with:

$$VOC = Wv - Ww$$

The results of the analysis are summarized below in Table 16:

TABLE 16
VOC content of samples.

| | | | | Weigh | | % | | | |
|---|---|---|---|---|---|---|---|---|---|
| | | | Sample | dish | | Water | | % | % |
| | | Weigh dish, | weigh, | after, | % | by | % | VOC | VOC |
| Sample ID | Chemistry | g | g | g | Evaporate | weigh | VOC | average | reduce |
| Comparative | IPA 70% | 2.95798 | 3.01659 | 2.95792 | 100.002 | 35.28582 | 64.71617 | 64.71006 | 0 |
| Sample Z | | 3.02467 | 3.02494 | 3.02498 | 99.990 | 35.28582 | 64.70394 | | |
| Sample B | 4% H2O2 | 3.02322 | 3.02076 | 3.02423 | 99.96656471 | 56 | 43.96656 | 43.98018 | 32.06248 |
| | 40% IPA | 3.00803 | 3.06275 | 3.00822 | 99.99379642 | 56 | 43.9938 | | |
| Sample A | 3% H2O2 | 2.94522 | 3.07831 | 2.94539 | 99.99447749 | 57 | 42.99448 | 42.9974 | 33.56456 |
| | 40% IPA | 2.94793 | 3.0774 | 2.94792 | 100.0003249 | 57 | 43.00032 | | |
| Comparative | 70% IPA | 2.97117 | 2.98082 | 2.97127 | 99.99664522 | 35.28582 | 64.71083 | 64.71201 | 0.008257 |
| Sample X | | 3.01884 | 3.01736 | 3.01887 | 99.99900575 | 35.28582 | 64.71319 | | |

Overall, the inventive Samples A and B had reduced VOC content compared to Comparative Examples X and Z.

### Additional Examples

The following exemplary examples are provided, the numbering of which is not to be construed as designating levels of importance:

Example 1 can include a sanitizing solution formulation comprising: isopropyl alcohol in a range from about 70% to about 90% by weight of a first part; and hydrogen peroxide in a range of about 6% to about 8% by weight or less of a second part; the first part and the second part having a ratio of about 6:4 to a ratio of about 4:6 by volume, wherein the first part and the second part are applied simultaneously to a surface to produce the sanitizing solution, and wherein the sanitizing solution has a residual mass content of less than about 0.4 mg/g when assessed at least 5 minutes following application to the surface.

Example 2 can include Example 1, wherein the isopropyl alcohol is about 75% to about 85% by weight of the first part.

Example 3 can include any of Examples 1-2, wherein the hydrogen peroxide is about 6% to about 7% by weight of the second part.

Example 4 can include any of Examples 1-3, wherein the first part and the second part are stored in separated portions of a single container.

Example 5 can include any of Examples 1-4, wherein the first part and the second part are applied simultaneously by a sprayer to the surface.

Example 6 can include any of Examples 1-5, wherein isopropyl alcohol comprises about 30% to about 45% of the sanitizing solution when the first part and the second part are applied simultaneously.

Example 7 can include any of Examples 1-6, wherein isopropyl alcohol comprises about 35% to about 40% of the sanitizing solution when the first part and the second part are applied simultaneously.

Example 8 can include any of Examples 1-7, wherein hydrogen peroxide comprises about 3% to about 4% of the sanitizing solution when the first part and the second part are applied simultaneously.

Example 9 can include any of Examples 1-8, wherein hydrogen peroxide comprises about 3.2% to about 3.5% of the sanitizing solution when the first part and the second part are applied simultaneously.

Example 10 can include any of Examples 1-9, wherein the sanitizing solution has a residual mass content of less than about 0.3 mg/g.

Example 11 can include any of Examples 1-10, wherein the sanitizing solution has a residual mass content of less than about 0.2 mg/g.

Example 12 can include any of Examples 1-11, wherein the sanitizing solution has a residual mass content of less than about 0.1 mg/g.

Example 13 can include any of Examples 1-12, wherein the sanitizing solution has a residual mass content of less than about 0.05 mg/g.

Example 14 can include any of Examples 1-13, wherein the sanitizing solution has a residual total organic carbon (TOC) content of less than about 0.1 mg/g.

Example 15 can include any of Examples 1-14, wherein the sanitizing solution has a residual TOC content of less than about 0.05 mg/g.

Example 16 can include any of Examples 1-15, wherein the sanitizing solution has a residual TOC of less than about 0.01 mg/g.

Example 17 can include any of Examples 1-16, wherein the sanitizing solution has a volatile organic carbon (VOC) content of less than about 50 wt. %.

Example 18 can include any of Examples 1-17, wherein the sanitizing solution has a VOC content of less than about 45 wt. %.

Example 19 can include any of Examples 1-18, wherein the sanitizing solution does not substantially degrade stainless steel, aluminum, polytetrafluoroelthylene, polyvinyl chloride, polypropylene, polycarbonate, polyethylene, polyvinylidene fluoride, or polyvinylidene difluoride when applied thereto.

Example 20 can include a sanitizing solution formulation comprising 40% isopropyl alcohol and 3% to 4% hydrogen peroxide, wherein the formulation has a volatile organic content (VOC) of less than about 50 wt. %, a mass residual content of less than about 0.3 mg/g, and a residual total organic carbon (TOC) content of less than about 0.1 mg/g.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

What is claimed is:

1. A sanitizing solution formulation comprising:
isopropyl alcohol in a range from about 70% to about 90% by weight of a first part; and
hydrogen peroxide in a range of about 6% to about 8% by weight or less of a second part;
the first part and the second part having a ratio of about 6:4 to a ratio of about 4:6 by volume,
wherein the first part and the second part are applied simultaneously to a surface to produce the sanitizing solution, and
wherein the sanitizing solution has a residual mass content of less than about 0.4 mg/g when assessed at least 5 minutes following application to the surface.

2. The sanitizing solution of claim 1, wherein the isopropyl alcohol is about 75% to about 85% by weight of the first part.

3. The sanitizing solution of claim 1, wherein the hydrogen peroxide is about 6% to about 7% by weight of the second part.

4. The sanitizing solution of claim 1, wherein the first part and the second part are stored in separated portions of a single container.

5. The sanitizing solution of claim 1, wherein the first part and the second part are applied simultaneously by a sprayer to the surface.

6. The sanitizing solution of claim 1, wherein isopropyl alcohol comprises about 30% to about 45% of the sanitizing solution when the first part and the second part are applied simultaneously.

7. The sanitizing solution of claim 6, wherein isopropyl alcohol comprises about 35% to about 40% of the sanitizing solution when the first part and the second part are applied simultaneously.

8. The sanitizing solution of claim 1, wherein hydrogen peroxide comprises about 3% to about 4% of the sanitizing solution when the first part and the second part are applied simultaneously.

9. The sanitizing solution of claim 8, wherein hydrogen peroxide comprises about 3.2% to about 3.5% of the sanitizing solution when the first part and the second part are applied simultaneously.

10. The sanitizing solution of claim 1, wherein the sanitizing solution has a residual mass content of less than about 0.3 mg/g.

11. The sanitizing solution of claim 10, wherein the sanitizing solution has a residual mass content of less than about 0.2 mg/g.

12. The sanitizing solution of claim 11, wherein the sanitizing solution has a residual mass content of less than about 0.1 mg/g.

13. The sanitizing solution of claim 12, wherein the sanitizing solution has a residual mass content of less than about 0.05 mg/g.

14. The sanitizing solution of claim 1, wherein the sanitizing solution has a residual total organic carbon (TOC) content of less than about 0.1 mg/g.

15. The sanitizing solution of claim 14, wherein the sanitizing solution has a residual TOC content of less than about 0.05 mg/g.

16. The sanitizing solution of claim 15, wherein the sanitizing solution has a residual TOC of less than about 0.01 mg/g.

17. The sanitizing solution of claim 1, wherein the sanitizing solution has a volatile organic carbon (VOC) content of less than about 50 wt. %.

18. The sanitizing solution of claim 17, wherein the sanitizing solution has a VOC content of less than about 45 wt. %.

19. The sanitizing solution of claim 1, wherein the sanitizing solution does not substantially degrade stainless steel, aluminum, polytetrafluoroelthylene, polyvinyl chloride, polypropylene, polycarbonate, polyethylene, polyvinylidene fluoride, or polyvinylidene difluoride when applied thereto.

20. A sanitizing solution formulation comprising 40% isopropyl alcohol and 3% to 4% hydrogen peroxide, wherein the formulation has a volatile organic content (VOC) of less than about 50 wt. %, a mass residual content of less than about 0.3 mg/g, and a residual total organic carbon (TOC) content of less than about 0.1 mg/g.
